(19) United States
(12) Patent Application Publication
(10) Pub. No.: US 2023/0410982 A1
(43) Pub. Date: Dec. 21, 2023

(54) Secure Storage, Access, And Display Of Medical Image Data

(71) Applicant: PacificMD Biotech, LLC, Henderson, NV (US)

(72) Inventors: Jetmir Palushi, Irvine, CA (US); Ashley Sikand, Las Vegas, NV (US); Brian Hunter Weeks, Henderson, NV (US)

(21) Appl. No.: 18/198,669

(22) Filed: May 17, 2023

Related U.S. Application Data
(60) Provisional application No. 63/343,405, filed on May 18, 2022.

(51) Int. Cl. G16H 30/20

(52) U.S. Cl. CPC G16H 30/20

(57) ABSTRACT

A medical imaging system is configured to receive and securely store medical image data for patients, while providing sufficient access to view, review, and access image data. A care provider uses a cloud imaging hub and security token to receive medical image data produced by an image data source. The hub associates an arbitrary identifier with the medical image data based on patient information and the security token. The patient information is locally stored on the hub and not transmitted to any other device or server, and is scrubbed from the device after the UID is generated. The UID serves as an anonymous identifier for any medical image data associated with the patient, and may also be used to identify and access anonymous medical image data. The UID may be provided to the patient as a QR code, and is usable by the patient to access their anonymized image data and/or provide such access to another care provider.

### PRIORITY

This application claims the priority of U.S. Provisional Patent Application 63/343,405, filed May 18, 2022, and titled “Secure Storage, Access, and Display of Medical Image Data,” the entire disclosure of which is hereby incorporated herein by reference.

### FIELD

The disclosed technology pertains to a system for securely storing and displaying medical image data.

### BACKGROUND

Secure storage and handling of medical data is a complex problem that includes both technical challenges and requirements as well as business process challenges and requirements. Often such data is highly sensitive and highly regulated, while also requiring some level of visibility and accessibility—whether by third party medical providers, insurers, or by the patient themselves. The result is a set of contradictory needs and requirements—the data must be stored and provided with the utmost level of security and confidence, while also being readily available to a number of interested parties. This is especially true of medical image data (e.g., CT, MRI, or other scans, as well as endoscopic or other medical image photography or videography), as patients often like to access and view such images themselves, and often need to share such images with specialists and other care providers beyond the provider that initially created the image data.

What is needed, therefore, is an improved system for storing, accessing, and displaying medical image data.

### BRIEF DESCRIPTION OF THE DRAWINGS

The drawings and detailed description that follow are intended to be merely illustrative and are not intended to limit the scope of the invention as contemplated by the inventors.

FIG. 1 is a high-level architecture diagram for an exemplary system configured to store and provide medical image data.

FIG. 2 is a flowchart of an exemplary set of high-level steps that could be performed with a system to store and provide medical image data.

FIG. 3 is a flowchart of an exemplary set of steps that could be performed with a system to commission a cloud imaging hub and security tokens for use.

FIG. 4 is a flowchart of an exemplary set of steps that could be performed with a system to create a cloud imaging repository for a patient.

FIG. 5 is a flowchart of an exemplary set of steps that could be performed with a system to receive, anonymize, and store image data.

FIG. 6 is a flowchart of an exemplary set of steps that could be performed with a system to provide image data to an origin site device.

FIG. 7 is a flowchart of an exemplary set of steps that could be performed with a system to provide image data to a patient device.

FIG. 8 is a flowchart of an exemplary set of steps that could be performed with a system to migrate image data to another site device.

FIG. 9A is a screenshot of an exemplary patient image data interface prior to de-anonymization.

FIG. 9B is a screenshot of an exemplary patient image data interface after de-anonymization.

### DETAILED DESCRIPTION

The inventors have conceived of novel technology that, for the purpose of illustration, is disclosed herein as applied in the context of secure storage, access, and display of medical image data. While the disclosed applications of the inventors' technology satisfy a long-felt but unmet need in the art of secure storage, access, and display of medical image data, it should be understood that the inventors' technology is not limited to being implemented in the precise manners set forth herein, but could be implemented in other manners without undue experimentation by those of ordinary skill in the art in light of this disclosure. Accordingly, the examples set forth herein should be understood as being illustrative only, and should not be treated as limiting.

Implementations of the disclosed technology provide a medical imaging system that is configured to receive and securely store medical image data for patients, while simultaneously providing sufficient access to such patients and/or other care providers to view, review, and access the image data. While conventional approaches to data security emphasize end-to-end platforms and processes, such approaches are both complex and, often as a result of such complexity, vulnerable to various technical and non-technical attacks (e.g., such as ransomware attacks, database mining, client-side spyware or malicious software, phishing attempts, spear fishing attempts, social engineering attacks, etc.). Such complexity also contributes to cost of development, cost of implementation for particular customers, cost and time required for customer training, and even costs of contract negotiation between a software provider and their customer hospital or clinic.

These shortcomings and others are addressed by implementations of the disclosed technology. Such implementations emphasize simplicity, minimize use of patient information, minimize the scope and footprint of the system within the greater context of health information systems (“HIS”) and medical care providers, and minimize provider and patient interactions with the system. As example of the above, a care provider (e.g., a hospital, hospital system, small clinic, etc.) uses a cloud imaging hub and corresponding security token that are commissioned specifically for that care provider (e.g., either for the care provider generally, or for a particular doctor or other professional within that care provider) to receive medical image data produced by an image data source (e.g., an endoscope, MRI or CT equipment).

The cloud imaging hub is configured with software to generate and associate an arbitrary identifier with the medical image data based on a set of patient information (e.g., name, birthdate, email address, phone number, and combinations thereof), and is configured to only generate the identifier when coupled to the security token (e.g., to enable access to/receipt of a whole or partial encoding or encryption key stored on the security token) and/or based upon information provided by the security token (e.g., such as a continuously expiring/refreshing key displayed or otherwise communicated by the security token). This arbitrary identifier may be an integer or a string of varying content, and is referred to herein as a unique identifier (“UID”). The patient information is confined to being locally stored on the cloud imaging hub and is not transmitted to any other device or server, and is scrubbed from the device entirely from the cloud imaging hub immediately after the UID is generated.

Once generated, the UID serves as an anonymous identifier for any medical image data associated with the patient, and may be associated with image data file names, directories, and metadata, and may be embedded within/displayed within the image data itself. In this manner, the UID determines the locations (e.g., databases, file structures, accessible via a web URL or software application) to which received image data is provided and stored on, and so also indicate where such information may be accessed by the care provider, the patient, and other care providers. The UID may be provided to the patient in varying ways (e.g., as a QR code, configured within a software application on a smartphone or other device, or as printed text), and is usable by the patient to access their anonymized image data and/or provide such access to another care provider.

The UID is not stored by the cloud imaging hub or related devices, but instead may be re-created by the cloud imaging hub based upon re-entering the patient information that was initially used to generate the UID, or may be decoded from the UID when the cloud imaging device is coupled to the security token, or the security token is otherwise available to use with the cloud imaging device. As an example, the cloud imaging device may provide access to view and navigate the anonymized contents stored on cloud imaging database, though such navigation is limited to viewing patient medical image data organized by the UID instead of identifiable information of the patient. When coupled to the security token, the cloud imaging device may locally identify UIDs that are received from the cloud imaging database, and may use the UID software and keys on the imaging device and security token to decode those UIDs and convert them into identifiable patient information in real time, and entirely locally to the cloud imaging device (e.g., without sending/transmitting any patient information, or any decoding/decryption keys or other information, to any external device). Once this real time determination is made, a local interface layer may be overlaid or applied to the anonymized medical image data listing in order to associate patient information with the medical image data, and to assist the care provider in their location and review of medical image data.

Access to the medical image database may be provided to patients and/or other care providers by lookup of the anonymous UID (e.g., provided as a QR code, link, string text, or via a software application configured to store one or more UIDs) via a website or software application, though without the properly configured and commissioned cloud imaging device and security token used to upload the medical imaging data, it will remain anonymous (e.g., de-identified medical image data may be viewable in some cases, but will not contain any patient identifiable information). Additional safeguards may be taken to prevent iterative/randomized access to medical image data, such as a user login, two factor authentication, or other authentication process, or by monitoring and blocking suspicious activity by IP addresses or other network identifiers.

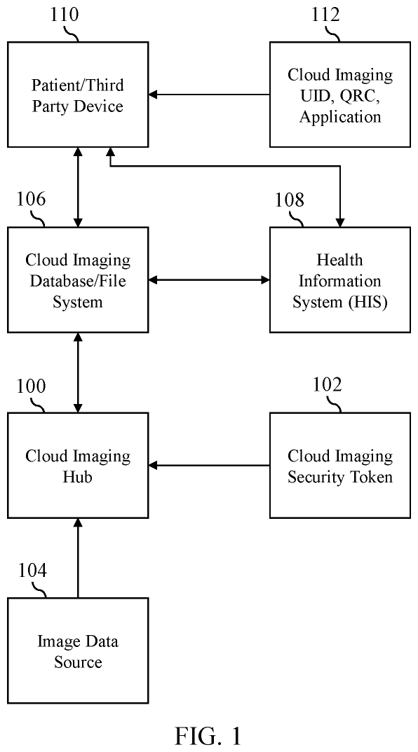

FIGS. 1 through 9B provide additional examples and illustration of the above. FIG. 1 is a high-level architecture diagram for an exemplary system configured to store and provide medical image data. A cloud imaging hub (100) may be a computer, tablet computer, smartphone, laptop, or other computing device having a display, user interface, processor and memory, and other common computer components. The cloud imaging hub may be configured with a specialized operating system or software application that is pre-configured to receive, anonymize, and upload image data, and that is configured with a UID generation (e.g., usable for UID encoding and UID decoding) software that is commissioned for the site and corresponding security tokens. A cloud imaging security token (102) may be an electronic memory fob (e.g., a USB memory, NFC or RFID memory, or other memory device, or a security token usable to display or otherwise provide a full or partial security key or other information) configured to store information corresponding to the UID generation software, and configured to enable the operation of the UID generation software.

An image data source (104) may be an endoscope, MRI or CT system, or other system producing or providing medical image data to the cloud imaging hub (100). A cloud imaging database (106) may be a cloud repository such as a file system, database, or other high volume cloud enabled storage device, and is configured to receive and store medical image data from the cloud imaging hub (100), and to provide or display anonymized medical image data (e.g., via a web browser or web application, or other software application). In some implementations, a care provider clinic or other location may configure a health information system (“HIS”) (108) to communicate with the cloud imaging database (106) in order to download certain medical image data. While not required, the HIS may be configured by the user with additional data, such as to associate UIDs with certain patients that have other records in the HIS (108). However, this is not required by the cloud imaging hub (100), and would be solely at the option (and potential security risk) of the administrator of the HIS (108).

A patient or other third party (110) may be a computer, tablet computer, smartphone, or other computing device usable to access and view medical image data stored on the cloud imaging database (106). Such access will typically be limited to viewing anonymized medical image data. A cloud imaging UID (112) may correspond to the UID generated by the cloud imaging hub (100) when medical image data is uploaded, and may be provided to the patient and/or usable with the patient device (110) as a printed text string, QR code, RFID code, electronic link, or other information usable by the patient to access and view anonymized medical image data on the cloud imaging database (106).

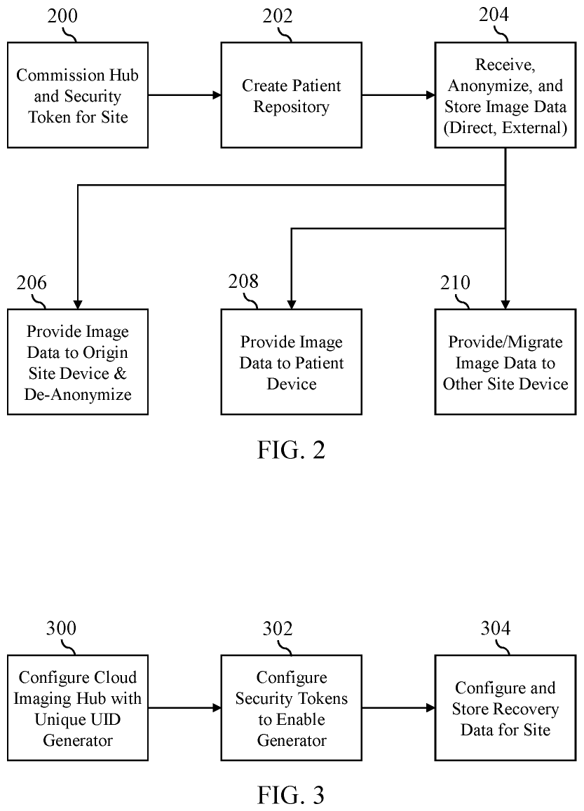

FIG. 2 is a flowchart of an exemplary set of high-level steps that could be performed with a system to store and provide medical image data. As has been described above, the system may be used to commission (200) and configure the cloud imaging hub and security tokens for a particular site, care provider system, or for individual care providers. Commissioning (200) may include providing and assigning unique encoding/decoding keys or other information usable to ensure that different sets of cloud imaging hub and security tokens (e.g., commissioned for different sites) generate different UIDs for identical patient information.

The system may also create (202) a patient repository on the cloud database (106) for each set of received patient information and based on the UID generated from that patient information. The system may also receive (204), anonymize, and store image data in the created (202) repository, and such image data may be received directly from imaging instruments (e.g., endoscopes, MRI equipment) or may be received from a non-imaging source (e.g., a patient provided USB drive or other memory, via FTP or other data transfer from another clinic providing care to the patient).

The system may also provide (206) stored and anonymized image data to devices at the origin site of such data, in which case the image data may be de-anonymized and viewed in the context of patient information. The system may also provide (208) image data to patients or other requesters as anonymized image data, such as via a web browser loading a UID based web location, or another software application. The system may also provide (210) medical image data to another user of the system as migrated data, which may include mapping or defining logical storage addresses to a separate UID for the patient that is specific to the recipient site (e.g., the commissioned hub and security tokens of the recipient site). In this manner, a single file dataset may be stored in the cloud database (106) that may resolve to separate UIDs for separate clinics or care providers that are treating a shared patient.

FIG. 3 is a flowchart of an exemplary set of steps that could be performed with a system to commission a cloud imaging hub and security tokens for use. The system may be used to configure (300) the cloud imaging hub with a uniquely configured UID generator software application, which is operable to generate globally/universally unique UIDs based on patient information, when used with the corresponding security token. The system may also be used to configure (302) one or more security tokens with stored information corresponding to the cloud imaging hub, and configured to enable operation of the unique UID generator. The system may also configure (304) and store recovery data for each set of commissioned cloud imaging hub(s) and security token(s), which may include storing unique data usable to commission additional sets of devices in the future in the event that a site's hubs and/or security tokens become lost or unusable. This may include storing drive images that may be loaded to the device, or may include unique keys or codes usable to rebuild the commissioned device software, such that any previously created UIDs may still be encoded and decoded. Stored (304) recovery data is not stored or accessible on the cloud database (106), and instead may be securely stored offline or archived, and may only be used to rebuild or commission new devices as a manual process through customer or administrative services associated with the system, and only after thorough and/or on-site identity verification.

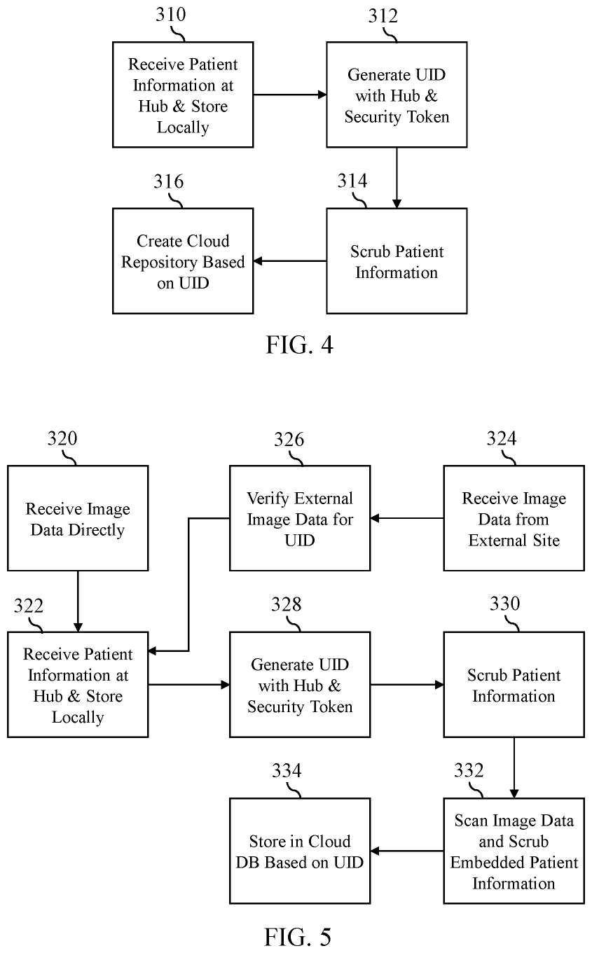

FIG. 4 is a flowchart of an exemplary set of steps that could be performed with a system to create a cloud imaging repository for a patient. The system may receive (310) patient information for a new or returning patient and may store that information locally on the imaging hub. The information may be stored in a temporary memory or cache, and never written to a persistent storage device, or transmitted to an external device. The information may be received via manual input and/or by interfacing with the site's HIS (108), where available. The imaging hub may then generate (312) a UID based on the patient information (e.g., by encoding the patient information into an arbitrary, but decodable, string or other data), its own configuration, and the configuration or received information from a corresponding security token. Once the UID is generated (312) the imaging hub may scrub (314) all received patient information from any memory that stored it upon receipt, and based on the UID may create (316) or, in the case of a returning patient, confirm the existence of, a cloud repository location to receive and store the medical imaging data.

FIG. 5 is a flowchart of an exemplary set of steps that could be performed with a system to receive, anonymize, and store image data. The system may receive (320) medical image data directly from imaging equipment such as an endoscope, MRI or CT equipment, or another device. The system may also receive (322) patient information corresponding to that medical image data, as described above in the context of FIG. 4 (e.g., manual input, received from HIS (108), received with medical image data from image source equipment). In some implementations, the system may also receive (324) medical image data for a patient from an external source such as an HIS (108) of another care provider site or facility. Received (324) external data may be subject to verification (326), either automated or manual review and verification, or both, and may include verifying that the patient information matches an existing UID repository, or matches existing patient records in the HIS (108), for example. Such information is also received (322) with corresponding patient information.

Regardless of origin, the system may generate (328) a UID based on the patient information and using the commissioned hub and security token, and may scrub (330) the patient information from any location on the hub device where it was stored, as described above in the context of FIG. 4. The system may also scan (332) the medical image data itself to identify and scrub any embedded patient information (e.g., such as full or partial patient information that might be associated with the image data as metadata, or visible in images or image models, or otherwise). Once fully anonymized, the system may store (334) the anonymized medical image data in the imaging database (106) based on the generated (328) UID, as has been described.

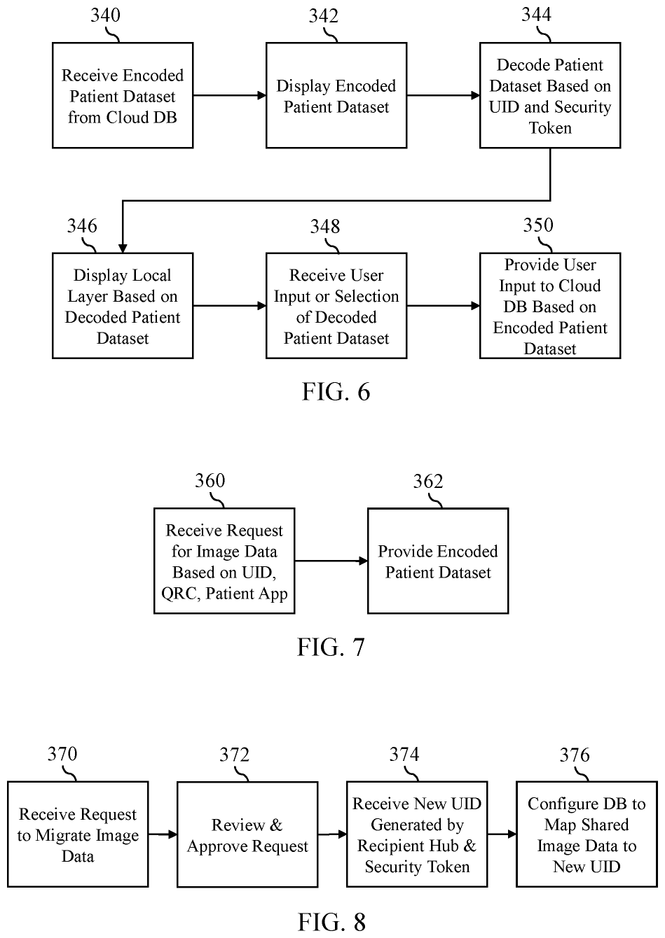

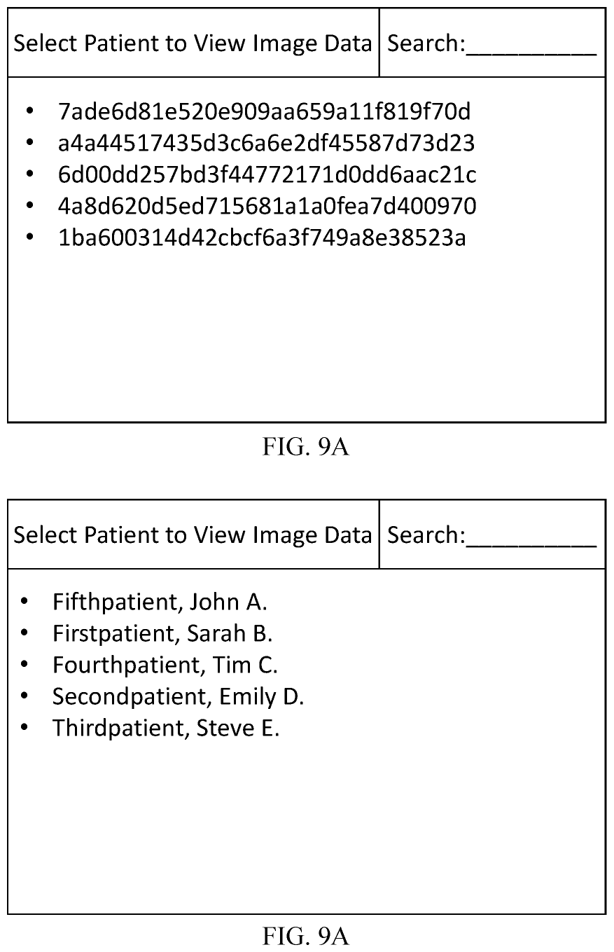

FIGS. 6 through 8 each provide examples of steps performed when providing image data to a requesting device. FIG. 6 is a flowchart of an exemplary set of steps that could be performed with a system to provide image data to an origin site device that originally anonymized and stored the image data (e.g., the commissioned hub device and security token at the site or facility where the image data was originally uploaded from). The origin site device may receive (340) an encoded patient dataset from the cloud database (106), for example as a web interface or software application interface, and may display (342) the encoded patient dataset. When initially received and/or displayed, or when received and displayed on the imaging hub without the corresponding security token, the encoded patent dataset is anonymized, but indicates the presence of medical image data associated with one or more UIDs. The displayed interface may be navigated (e.g., by clicking or scrolling) to select and view medical image data by UID, date of creation, or other non-identifiable information, but it is not navigable or searchable based upon patient information. As an example, FIG. 9A shows an exemplary interface that may be displayed on the cloud imaging hub based on an encoded patient dataset. Each of the five displayed UIDs may be clicked upon to view and or gain further information on medical image data, but patient information is not present or viewable.

Next, the system may decode (344) the patient dataset based on the UIDs present in the patient dataset, and the commissioned imaging hub and corresponding security tokens, to decode the UIDs into the patient information that was provided and used to originally generate and encode the UID. The system may then display (346) the decoded patient dataset as a local interface layer, with decoded patient information only being temporarily stored on memory, and not transmitted to any external device. The locally displayed layer may be displayed as a modification of the prior displayed layer (e.g., such as a client-side JavaScript process that finds and replaces the affected text) or may be a superimposed over the prior displayed layer (e.g., a client side JavaScript or other process may add additional interface elements over top of or proximate to the prior displayed elements, but those prior displayed elements may not actually be modified to remove, overwritten, or hidden from display).

As yet another example, the system may include an additional user device such as a head-mounted display configured to provide virtual reality or augmented reality displays to a user, or another device including a camera and display capable of capturing images and providing augmented reality displays (collectively, “augmented reality device”). The augmented reality device may be configured to capture images of the displayed (342) encoded dataset from a display of the hub device (100) or another device, and to decode (344) and display (346) the decoded layer locally via a display of the augmented reality device. The augmented reality device may be in communication with a device capable of decoding (344) the patient dataset, or may itself be configured to decide (344) the patient dataset (e.g., the augmented reality device may be configured to store and/or receive the commissioning data and configurations from one or both of the hub device (100) and security token (102)). Once decoded, the patient dataset may be displayed (346) locally as an augmented reality overlay (e.g., a virtual text, graphic, or other display elements positioned and displayed over the originally displayed text within an augmented reality three-dimensional space) via a head-mounted display, translucent over-eye display, or other display.

FIG. 9B shows an exemplary interface such as that of FIG. 9A, after the encoded patient dataset has been decoded (344) and the local layer has been displayed (346) by the client device, the imaging hub. As can be seen, each UID is replaced in near real time with the patient's name or other patient information, and the list of UIDs may be automatically or manually resorted based on alphabet or other sorting preferences, and may be searched or filtered based upon various patient information to find the desired records. Once the local layer is displayed (346), all decoded patient information may be scrubbed from local memory, such that the only patient information present is that displayed by the device (e.g., and stored in a corresponding display device memory or cache).

As a further example of the above, the encoded patient dataset may initially be displayed (342) as a website or web interface. The client device, the imaging hub, may locally decode (344) the patient dataset received as HTML, or streaming web data from the cloud database (106), and may locally modify the website display (e.g., using JavaScript or other client-side coding) to apply the local interface layer (346). Since this occurs entirely on the client device and independent of interaction with the cloud database (106), the patient information and commissioned device information used to decode the patient information is never shared or exchanged with any external device, however, the user of the client device is able to view and navigate the patient information and associated medical image data in a temporarily de-anonymized session.

The user may also interact with the displayed (346) local layer, such as by searching for certain patients, clicking on a patient entry to view medical image data, or otherwise. In such cases, the imaging hub may receive (348) such user input, and may translate (e.g., re-encode) any patient information or other previously encoded information before providing it to the cloud database (106) as an additional request. Once again in the form of an anonymized encoded patient dataset, the user input may be used to request additional information or resources from the cloud database (106) without exposing any patient information. Continuing the above web-based example, when clicking on a patient name to view that patient's repository of medical image data, the imaging hub may re-encode that patient name, or may modify any underlying navigation link, to ensure that it does not contain any information, and that it properly resolves to a UID on the cloud database (106), before providing the web request to the cloud database (106).

FIG. 7 is a flowchart of an exemplary set of steps that could be performed with a system to provide image data to a patient device. When receiving care, a patient may be provided with a printed UID or QR code, or an electronic copy of a UID or QR code, or an electronic memory such as an RFID containing their UID, or may be provided with a software application configurable on their smartphone that stores that patient's UID. The provided UID is usable by the patient to access their medical image data on the cloud database (106). In such a case, the system may receive (360) a request for image data from the patient's requesting device based on the provided UID, and may provide the patient with an encoded patient dataset associated with that patient's medical image data (e.g., a listing of image files or models) to be displayed on their device (e.g., as a website, software applications, etc., as has been described). In this manner, the patient is able to use their UID to access and view their medical image data, although it does remain anonymized. However, in the event that a bad actor or other party is able to gain access to the arbitrarily named image data repository, it will only contain anonymized medical image data, and so will be of little use to the recipient and will not compromise the security or privacy of the patient.

FIG. 8 is a flowchart of an exemplary set of steps that could be performed with a system to migrate image data to another site device. Where two different care providers share a common patient, and are each using an implementation of the disclosed system, there may be a need to migrate medical image data from one provider to another where the patient wishes for it to be shared. Since each care provider has a different uniquely commissioned set of hub device(s) and security token(s), the subsequent care provider will not be able to decode the UID that was generated by the original care provider, and so the medical image data cannot be de-anonymized. To address this, the system may receive (370) a request to migrate image data from the subsequent care provider, and an administrator of the original care provider may review (372) and approve the request based upon manual confirmation with the patient and/or subsequent care provider.

Once confirmed, the system may receive (374) a new UID for the patient information that is generated by the subsequent care provider (e.g., based upon their uniquely commissioned imaging hub(s) and security token(s)). The system may then configure (376) the imaging database (376) to map some or all of the patient's medical image data to be associated with the newly generated UID, in addition to the originally generated UID. In this manner, patients and care providers may selectively share or withhold certain medical imaging data where desired. Mapping (376) of the shared image data may include defining logical links such that a single image file dataset corresponds to multiple UID associated locations, file directories, or other information, allowing the two separate sites to access and use the data as described above, but without requiring the creation and storage of duplicate medical image data files.

Some implementations of the disclosed technology may include an analytics process (e.g., an expert module, machine learning process or other artificial intelligence function, or other analytics function) that runs continuously in the background (e.g., of the hub device (100) or cloud imaging database (106)) combining key performance indicators from multiple database sources and provides to each user a dashboard that includes real time update with key patterns that result in improved procedure outcomes. The AI algorithms or other analytics functions are, themselves updated from (e.g., configured based upon, trained by a training dataset produced from) the vast amount of the data generated into the cloud, and the optimal performing version of the application automatically is downloaded to the users at a pre-designated time the user can select to upgrade their software application. Furthermore, the analytics functions running can be configured to collect data around the health of each system operating at a clinic and in real time alerts the manufacturer when a problem is encountered with the hardware or software in the doctor's clinic (e.g., hardware failures of the imaging hub (100), security token (102), endoscope or other image data source, etc., as well as software and configurations stored and operating on the same).

In some implementations, the disclosed technology can be configured to track the data usage among all the clinical sites and patients (e.g., number of uses, amount of data stored in, uploaded to, or downloaded from the database (106). Such data may be used to enforce usage limitations and/or to automatically generate invoices and/or complete other transactions related to the system, and such invoicing and/or payments may be further adjusted as per use case, per data used, bundled with other products used from the same supplier (on which case the clinician can enter the serial number of the product being used), or at a fixed cost, for example.

It should be understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. A system for securely storing and providing medical image data comprising:
(a) a hub device comprising a processor, a storage device, and a display;
(b) a security token device configured to store a set of security token data, and configured to be selectively communicatively coupled to the hub device;
(c) a cloud imaging database that is in communication with the hub device and configured to store sets of medical image data;
wherein the processor is configured to:
(i) receive and locally store on the storage device a set of medical image data and a set of patient data, each associated with a patient, from an image data source;
(ii) access the set of security token data when the security token device is coupled to the hub device, and generate an anonymized identifier based on the set of patient data and the set of security token data;
(iii) create a patient repository for the patient on the cloud imaging database based on the anonymized identifier, and store the set of medical image data in the patient repository;
(iv) immediately after generating the anonymized identifier, scrub the locally stored set of patient data so that it is no longer stored on the storage device;
(v) display a patient image data interface via the display that comprises descriptions of a plurality of patient repositories stored by the cloud imaging database, wherein:
(A) when the set of security token data is not accessible by the hub device, the plurality of patient repositories are each displayed based upon their corresponding anonymized identifiers;
(B) when the set of security token data is accessible by the hub device:
(I) the processor is configured to convert each anonymized identifier to a corresponding set of patient data based upon the set of security token data; and
(II) the plurality of event repositories are each displayed based upon their corresponding set of patient data.

2. The system of claim 1, wherein the processor is further configured to, when displaying the patient image data interface with the set of security token data accessible by the hub device:
(a) display the plurality of patient repositories based upon their corresponding anonymized identifiers as a local display layer;
(b) access the set of security token data on the security token device, and search the local display layer to identify each anonymized identifier; and
(c) convert each anonymized identifier to the corresponding set of patient based on the set of security token data, and update the local display later to show the corresponding sets of patient data.

3. The system of claim 2, wherein the corresponding sets of patient data:
(a) are not stored in any persistent storage device of the hub device;
(b) are not provided to any other device by the hub device; and
(c) are stored by the hub device only while the patient image data interface is displayed.

4. The system of claim 1, wherein the security token device is a portable electronic memory device.

5. The system of claim 1, wherein the processor is further configured to create an optical code for the patient based on the location of the patient repository in the cloud imaging database, wherein the optical code is configured to be read by a user device of the patient to cause the user device of the patient to access the patient repository.

6. The system of claim of claim 1, wherein the processor is further configured to, before storing the set of medical image data in the patient repository:
(a) scan the set of medical image data to identify any embedded patient data; and
(b) modify the set of medical image data so that the embedded patient data is not present.
